(19) United States
(12) Patent Application Publication
(10) Pub. No.: US 2023/0034357 A1
(43) Pub. Date: Feb. 2, 2023

(54) Aerial Disinfection

(71) Applicant: Grignard Pure LLC, Rahway, NJ (US)

(72) Inventor: Emile Etienne Grignard, III, Long Branch, NJ (US)

(21) Appl. No.: 17/933,365

(22) Filed: Sep. 19, 2022

Related U.S. Application Data
(63) Continuation of application No. PCT/US2021/030881, filed on May 5, 2021.
(60) Provisional application No. 63/166,074, filed on Mar. 25, 2021.
(60) Provisional application No. 63/113,632, filed on Nov. 13, 2020.
(60) Provisional application No. 63/020,395, filed on May 5, 2020.

(51) Int. Cl. A01N 31/02; A61L 2/22; A61L 2/20; A61L 9/14; A61L 9/02; A61L 9/04; F24F 8/24; A01N 25/18; A01P 1/00

(52) U.S. Cl. CPC A01N 31/02; A61L 2/22; A61L 2/20; A61L 9/14; A61L 9/02; A61L 9/044; F24F 8/24; A01N 25/18; A01P 1/00; A61L 2101/38

(57) ABSTRACT

This disclosure relates to a method for disinfecting a space. The method includes spraying a composition containing triethylene glycol into a space containing severe acute respiratory syndrome coronavirus 2 (SARS-CoV-2) in an amount effective to inactivate SARS-CoV-2.

### CROSS-REFERENCE TO RELATED APPLICATIONS

The present application is a continuation of and claims priority to International Application No. PCT/US2021/30881, filed on May 5, 2021, which claims priority to U.S. Provisional Application Ser. No. 63/166,074, filed on Mar. 25, 2021, U.S. Provisional Application Ser. No. 63/113,632, filed on Nov. 13, 2020, and U.S. Provisional Application Ser. No. 63/020,395, filed on May 5, 2020. The contents of the parent applications are hereby incorporated by reference in their entirety.

### TECHNICAL FIELD

This disclosure relates to compositions and methods for disinfecting a space (e.g., an indoor or outdoor space).

### BACKGROUND OF THE DISCLOSURE

Coronavirus disease COVID-19 is an infectious disease caused by a newly discovered coronavirus, i.e., severe acute respiratory syndrome coronavirus 2 (SARS-CoV-2). It is believed that SARS-CoV-2 is mainly transmitted through droplets generated when an infected person coughs, sneezes, or exhales. These droplets may fall on floors or surfaces, or may be in the form of an aerosol suspending in the air.

COVID-19 has caused a worldwide pandemic that has infected more than 3.5 million people and caused more than 250K deaths as of April, 2020. Thus, there is a great need to develop a method to stop the COVID-19 pandemic.

### SUMMARY OF THE DISCLOSURE

It has been found that SARS-CoV-2 is most stable at a relatively low humidity (e.g., 20% or less), a relatively low temperature, or in a dark area without direct sunlight. Thus, it is believed that SARS-CoV-2 poses the greatest risk in an indoor space or environment. The inventor has surprisingly found that spraying (e.g., by atomizing through an atomizing nozzle) a composition containing triethylene glycol (TEG) can effectively inactivate SARS-CoV-2 in the air and/or on the surfaces in an indoor space, thereby effectively disinfecting the indoor space (i.e., occupied or unoccupied by human being).

In one aspect, this disclosure features a method for disinfecting a space, the method including spraying a composition containing triethylene glycol into a space containing SARS-CoV-2 in an amount effective to inactivate SARS-CoV-2.

In another aspect, this disclosure features a composition that includes triethylene glycol in an amount of from about 52% to about 90% by weight of the composition; deionized water in an amount of from about 5% to about 48% by weight of the composition; and propylene glycol in an amount of from about 0% to about 5% by weight of the composition.

In another aspect, this disclosure features a packaged product that includes a container, and the composition described herein in the container.

Other features, objects, and advantages will be apparent from the description and the claims.

### DETAILED DESCRIPTION OF THE DISCLOSURE

As defined herein, unless otherwise noted, all percentages expressed should be understood to be percentages by weight to the total weight of a composition.

In general, this disclosure relates to compositions and methods for disinfecting a space (e.g., an indoor space), such as those in offices, schools, hotels, lobbies, theaters, reception rooms, bathrooms, health care facilities (e.g., nursing homes, hospital rooms (e.g., intensive care facilities), and medical offices (e.g., dental offices)), institutional kitchens, cafeterias, restaurants, public transportation vehicles (buses, trains, subways, and airplanes), ambulances, indoor stadiums and athletic facilities, law enforcement facilities (e.g., prisons), government facilities, elevators, retail locations, and other indoor public spaces.

In some embodiments, this disclosure features a disinfecting composition containing (e.g., comprising, consisting essentially of, or consisting of) triethylene glycol and water (e.g., deionized water). Triethylene glycol is miscible with water, has a boiling point of 286.5° C. at a pressure of 101.325 kPa, and has a relative low vapor pressure compared to water. Without wishing to be bound by theory, it is believed that triethylene glycol is highly hygroscopic and inactivates SARS-CoV-2 by condensing on virus-containing particles, droplets, or surfaces until the concentration of triethylene glycol becomes sufficiently high to break down the virus. In addition, without wishing to be bound by theory, it is believed that triethylene glycol has very low acute or chronic toxicity when inhaled or ingested (especially at the level used in the air to disinfect an indoor space) and therefore is safe to use in indoor spaces.

In general, the amount of triethylene glycol in the disinfecting composition described herein is not particular limited and can vary as desired. For example, a disinfecting composition containing a relatively low amount of triethylene glycol can achieve the same disinfection effect as a disinfecting composition containing a relatively high amount of triethylene glycol by spraying the former composition in an indoor space at a higher frequency or in a higher amount. In some embodiments, the disinfecting composition described herein can include triethylene glycol in an amount of from at least about 10% (e.g., at least about 20%, at least about 30%, at least about 40%, at least about 50%, at least about 52%, at least about 55%, at least about 60%, at least about 65%, at least about 70%, at least about 75%, or at least about 80%) by weight to at most about 90% (e.g., at most about 85%, at most about 80%, at most about 75%, at most about 70%, at most about 65%, at most about 60%, or at most about 50%) by weight of the composition. In some embodiments, triethylene glycol can be 100% of the disinfecting composition described herein (i.e., without any other ingredient). It is believed that spraying a disinfecting composition containing a relatively high amount (e.g., at least about 50% by weight) of triethylene glycol can increase the efficiency of the disinfection and reduce the frequency of the application of the composition.

In some embodiments, the water in the disinfecting composition described herein is deionized water. For example, the deionized water can include ions in an amount of from at most about 50 ppm (e.g., at most about 40 ppm, at most about 30 ppm, at most about 20 ppm, at most about 10 ppm, at most about 5 ppm, or at most about 1 ppm) to at least about 1 ppb (e.g., at least about 10 ppb) of the total amount of the deionized water.

In some embodiments, the disinfecting composition described herein can include deionized water in an amount of from at least about 5% (e.g., at least about 10%, at least about 15%, at least about 20%, at least about 25%, at least about 30%, at least about 35%, at least about 40%, at least about 45%, at least about 48%, at least about 50%, at least about 60%, or at least about 70%) by weight to at most about 90% (e.g., at most about 85%, at most about 80%, at most about 75%, at most about 70%, at most about 65%, at most about 60%, at most about 50%, or at most about 48%) by weight of the composition. Without wishing to be bound by theory, it is believed that deionized water can minimize clogging the nozzles (e.g., caused by deposition of minerals in water) of the system used to spray the disinfecting composition described herein and therefore can keep the system operating for an extended period of time. In addition, without wishing to be bound by theory, it is believed that the water in the disinfecting composition described herein can facilitate inactivation of SARS-CoV-2 as SARS-CoV-2 is less stable in a humid environment.

Without wishing to be bound by theory, it is believed that including water in the disinfection composition can allow the composition to be readily atomized (e.g., by a humidifier, a fog/haze machine, or a smoke generator) and to form an aerosol in the atmosphere. The water in the aerosol can evaporate rapidly to form fine TEG droplets, that have disinfect effects and kill SARS-CoV-2 in the air. In addition, the water in the disinfection composition can render the composition inflammable, thereby resulting in a safer product than TEG alone (which is a flammable liquid having a flash point of 157° C.).

In some embodiments, the disinfecting composition described herein can further include an optional ingredient, such as a glycol different from triethylene glycol. In some embodiments, the additional glycol can be a propylene glycol or a polyethylene glycol. As used herein, the “polyethylene glycol” refers to those have at least four ethylene oxide units. In some embodiments, the polyethylene glycol has a number average molecular weight of at least about 200 g/mol (e.g., at least about 300 g/mol, at least about 400 g/mol, at least about 500 g/mol, at least about 1000 g/mol) to at most about 4000 g/mol (e.g., at most about 3500 g/mol, at most about 3000 g/mol, at most about 2500 g/mol, or at most about 2000 g/mol). Without wishing to be bound by theory, it is believed that the additional glycol can either increase the disinfecting effect of the composition or increase the whiteness of the composition (e.g., to indicate that the disinfecting composition is present in the air). In some embodiments, the disinfecting composition described herein does not include any additional glycol or any components other than triethylene glycol and water.

In some embodiments, the disinfecting composition described herein can include an additional glycol (e.g., a propylene glycol or a polyethylene glycol) in an amount of from at least about 0.5% (e.g., at least about 1%, at least about 1.5%, at least about 2%, at least about 2.5%, at least about 3%, at least about 3.5%, at least about 4%, at least about 4.5%) by weight to at most about 5% (e.g., at most about 4.5%, at most about 4%, at most about 3.5%, at most about 3%, at most about 2.5%, at most about 2%, at most about 1.5%, or at most about 1%) by weight of the composition.

In some embodiments, the disinfecting composition described herein can include (e.g., comprise, consist essentially of, or consist of) from about 50% to about 90% by weight triethylene glycol and from about 10% to about 50% by weight deionized water. In some embodiments, the disinfecting composition described herein can include (e.g., comprise, consist essentially of, or consist of) (1) triethylene glycol in an amount of from about 52% to about 90% by weight of the composition; (2) deionized water in an amount of from about 5% to about 48% by weight of the composition; and (3) propylene glycol in an amount of from about 0% to about 5% (e.g., from about 0.5% to about 5%) by weight of the composition. In some embodiments, the disinfecting composition described herein can include (e.g., comprise, consist essentially of, or consist of) about 52.5% by weight triethylene glycol, about 1% by weight propylene glycol, and about 46.5% by weight deionized water.

In some embodiments, this disclosure also features a method disinfecting a space (e.g., an indoor or outdoor space). In some embodiments, the method can include spraying (e.g., by atomizing) a composition containing triethylene glycol (e.g., the disinfecting composition described herein) into a space (e.g., an indoor space) containing SARS-CoV-2 in an amount effective to inactivate SARS-CoV-2. In some embodiments, the spraying can be performed by a system that generates fog, smoke, or haze, such as a humidifier or a smoke generator. The smoke generator can be those known in the art, such as the fog/haze machines or smoke simulators used emergency training or used in the lighting industry to generate theatrical effects. In some embodiments, such a system can have a liquid reservoir and can use an electric pump to propel the disinfecting composition in the liquid reservoir into a heat exchanger where the disinfecting composition is vaporized. The heated vapor is forced through a nozzle (e.g., an atomizing nozzle) as vapor and as liquid droplets (or liquid particles) that form an opaque fog, smoke, or haze. In some embodiments, the system (e.g., a humidifier) can generate a fog, smoke, or haze by using ultrasound, steam, and/or atomization.

In some embodiments, the spraying of the composition can form vapor and/or liquid droplets (or liquid particles) that contain triethylene glycol. In some embodiments, the liquid droplets can form an aerosol that contains triethylene glycol. In some embodiments, the aerosol liquid droplets can have an average diameter of from at least about 10 nm (e.g., at least about 20 nm, at least about 50 nm, at least about 100 nm, at least about 200 nm, at least about 500 nm, at least about 1 μm, at least about 2 μm, or at least about 5 μm) to at most about 10 μm (e.g., at most about 8 μm, at most about 6 μm, at most about 5 μm, at most about 4 μm, at most about 2 μm, at most about 1 μm). In some embodiments, the method described herein can generate from at least about 2000 (e.g., at least about 3000, at least about 4000, at least about 5000, at least about 6000, at least about 8000, or at least about 10,000) to at most about 50,000 (e.g., at most about 25,000) liquid droplets per cm³ of the space (e.g., the indoor space).

In some embodiments, the spraying can be performed continuously or intermittently (e.g., either at a constant interval or at irregular intervals). In some embodiments, when the spraying is performed intermittently at a constant interval, the frequency of the spraying can vary as desired depending on factors such as the concentration of triethylene glycol in the composition, the temperature and humidity of the space, the size of the space, the desired concentration of the composition in the space, and the air exchange rates. In some embodiments, the preferred temperature of the space can range from about 10° C. to about 50° C. (e.g., from about 15° C. to about 30° C.). In some embodiments, the preferred relative humidity of the space can range from about 30% to about 65% (e.g., from about 45% to about 60%). In some embodiments, the time period between two sprayings can be from at least about 5 minutes (e.g., at least about 10 minutes, at least about 20 minutes, at least about 30 minutes, or at least about 1 hour) to at most about 3 hours (e.g., at most about 2.5 hours, at most about 2 hours, at most about 1.5 hours, or at most about 1 hour).

In some embodiments, the concertation of the disinfecting composition in a space can be from at least about 0.1 mg/m³ (e.g., at least about 0.2 mg/m³, at least about 0.3 mg/m³, at least about 0.4 mg/m³, at least about 0.5 mg/m³, at least about 0.6 mg/m³, at least about 0.8 mg/m³, at least about 1 mg/m³, at least about 1.5 mg/m³, at least about 2 mg/m³, at least about 2.5 mg/m³, at least about 3 mg/m³, at least about 3.5 mg/m³, at least about 4 mg/m³, at least about 4.5 mg/m³, or at least about 5 mg/m³) or at most about 10 mg/m³ (e.g., at most about 9.5 mg/m³, at most about 9 mg/m³, at most about 8.5 mg/m³, at most about 8 mg/m³, at most about 7.5 mg/m³, at most about 7 mg/m³, at most about 6.5 mg/m³, at most about 6 mg/m³, at most about 5.5 mg/m³, or at most about 5 mg/m³). For example, the concertation of the disinfecting composition in a space can be from at least about 0.3 mg/m³ to at most about 1.6 mg/m³.

In some embodiments, the concentration of the TEG in a space can be from at least about 0.05 mg/m³ (e.g., at least about 0.1 mg/m³, at least about 0.2 mg/m³, at least about 0.3 mg/m³, at least about 0.4 mg/m³, at least about 0.5 mg/m³, at least about 0.6 mg/m³, at least about 0.8 mg/m³, at least about 1 mg/m³, at least about 1.5 mg/m³, at least about 2 mg/m³, at least about 2.5 mg/m³, at least about 3 mg/m³, at least about 3.5 mg/m³, at least about 4 mg/m³, at least about 4.5 mg/m³, or at least about 5 mg/m³) or at most about 10 mg/m³ (e.g., at most about 9.5 mg/m³, at most about 9 mg/m³, at most about 8.5 mg/m³, at most about 8 mg/m³, at most about 7.5 mg/m³, at most about 7 mg/m³, at most about 6.5 mg/m³, at most about 6 mg/m³, at most about 5.5 mg/m³, or at most about 5 mg/m³). For example, the concertation of the TEG in a space can be from at least about 0.2 mg/m³ to at most about 1.5 mg/m³.

Without wishing to be bound by theory, it is believed that the disinfecting composition or the TEG having a concentration within the above ranges can effectively kill or inactivate at least 98% (e.g., at least 98.5%, at least 99%, at least 99.5, or at least 99.9%) SARS-CoV-2 in a space within a short period of time (e.g., at most 60 minutes, at most 30 minutes, at most 15 minutes, at most 3 minutes, at most 1 minute, or at most 30 seconds). In some embodiments, at least about 0.5 oz. (e.g., at least about 1 oz. or at least about 2 oz.) of the disinfecting composition can be used in a space having a volume of 1000 cubic feet every 4 hours (e.g., every two hours or every one hour).

In some embodiments, the method can further include vaporizing the composition (e.g., in a humidifier, a fog/haze machine, or a smoke generator) before spraying the composition. In some embodiments, vaporizing the composition can be performed by treating the composition with ultrasonication, atomization, steam, or heating. For example, when vaporizing the composition is performed by ultrasonication, the method can include treating the composition with sound energy at an ultrasonic frequency (e.g., at least about 20 kHz) by using a sonicator (e.g., having an ultrasonic probe) to agitate the composition to generate vapor and liquid droplets, which can then pass through a nozzle to create an opaque fog, smoke, or haze. As another example, when vaporizing the composition is performed by heating, the method can include delivering the composition to a heat exchanger to vaporize the composition. The heated vapor can be forced through a nozzle as vapor and liquid droplets (or liquid particles) to form an opaque aerosol, fog, smoke, or haze.

In some embodiments, to practice the disinfecting method described herein in an indoor space, one can place a system described herein in the center or on one or more sides of the indoor space to be treated. In some embodiments, multiple systems can be used at appropriate places to ensure even haze distribution. The disinfecting composition described herein can be sprayed from the system(s) into the indoor space until a desired haze or disinfection level is achieved. In some embodiments, the spraying can continue intermittently (e.g., every 30 minutes) to maintain the haze or disinfection level.

In some embodiments, the disinfecting composition described herein can be applied to an indoor space to be treated via an HVAC unit. For example, a system containing the composition described herein can be connected to the return plenum of an HVAC unit through a tubing. The composition can then be sprayed into the indoor space through the HVAC unit until a desired haze or disinfection level is achieved. This approach can disinfect both the filter in the HVAC unit and the indoor space.

In some embodiments, the space (e.g., the indoor space) to be treated can include SARS-CoV-2 suspending in the air and the disinfection method described herein is capable of inactivating the SARS-CoV-2 in the air. In some embodiments, the space can include SARS-CoV-2 on a surface (e.g., either a hard or soft surface, or either a non-porous or a porous surface) and the disinfection method described herein is capable of inactivating the SARS-CoV-2 on the surface. In some embodiments, the surface can be any surface in an indoor or outdoor space, such as a surface of a wall, a floor, a desk, a chair, a computer, a rug, or a drape. Without wishing to be bound by theory, it is believed that triethylene glycol can adhere to SARS-CoV-2 either in the air or on a surface to inactivate the virus by breaking down or disrupt the protein or membrane of the virus.

In another aspect, this disclosure features a packaged product that includes a container (e.g., a can or a bottle), and the disinfecting composition described herein in the container. The packaged product can be either pressurized or non-pressurized.

The following examples are illustrative and not intended to be limiting.

### Example 1: Evaluation of a Disinfecting Composition for its Efficacy Against an Enveloped Virus on a Hard Non-Porous Surface

### Test Parameters

Disinfecting Composition: Triethylene glycol (52.25 wt %), propylene glycol (1 wt %), and DI water (46.75%). Specific Gravity at 20° C. is 1.07-1.09. Boiling point is 103.8° C. Freeze point is −29.3° C. Vapor pressure is 0.13 psi at 25° C. Kinematic viscosity is 13.96 cSt at 40° C. The composition is non-flammable.

Dilution of Disinfecting Composition: No dilution

### Virus: Human Coronavirus, ATCC VR-740, Strain 229E

Exposure Times: 15 minutes and 3 hours
Exposure Temperature: 25-29° C. (prior to use in testing, the room was brought to temp before turning off the air handling system, Start: 24.70° C., 15 minutes: 23.99° C., 3 hours: 23.50° C.)
Exposure Humidity: Start: 45.03%, 15 minutes: 62.93%, 3 hours: 66.65%
Organic Soil Load: 1% fetal bovine serum
Test Medium: Minimum Essential Medium (MEM) supplemented with 2% (v/v) heat-inactivated fetal bovine serum, 100 units/ml penicillin, 10 μg/ml gentamicin, and 2.5 μg/ml amphotericin B.

Indicator Cell Cultures: WI-38 (human lung) cells.

### Device Preparation

Hurricane 1800 FLEX (Chauvet DJ, Sunrise, Fla.) was used in this test. Prior to using the machine, the initial weight of the test disinfecting composition was measured. The Hurricane 1800 FLEX was placed on the floor of the testing room (˜104 m³), in a complete horizontal position (per page 3 in User Manual) at a distance of ˜5 feet from the low level carrier placement. The Hurricane 1800 FLEX was plugged into the outlet and then plugged in the wired timer controller to the remote connector socket on the back of the test device (per page 10 in User Manual).

The Hurricane 1800 FLEX was allowed to heat up for three to five minutes. With the fluid intake tube in the test substance bottle, the Manual button on the remote control was pressed in order to prime the machine. The test disinfecting composition was then weighed again following the priming and returned to the Hurricane 1800 FLEX for use in testing. The amounts of the test disinfecting composition at various stages are listed below.

### Pre Test: 4175.72 g

### Post Prime: 4162.21 g

### Post Test: 4092.23 g

### Input Virus Control

On the day of test, the stock virus utilized in the assay was titered by 10-fold serial dilution and assayed for infectivity to determine the starting titer of the virus. The results of this control are for informational purposes only.

### Contamination of Carriers

The carriers used in this experiment were 100 mm×15 mm glass petri dishes. For each carrier, a 200 μL aliquot of test virus was added to the surface of a carrier. The virus was air-dried at 10° C.-30° C. until visibly dry (20 minutes). The drying conditions (temperature and humidity) were appropriate for the test virus for the purpose of obtaining maximum survival following drying.

### Testing

Prior to the start of testing, the test room was at a temperature of 25-29° C. The air system was turned off at the start of testing to prevent interference with the test device. In order to attempt to maintain a relative humidity of 45-65% in the test room, a humidifier was added to the testing room. The temperature and relative humidity were measured and listed below.

Temperature: Start: 24.70° C., 15 minutes: 23.99° C., 3 hours: 23.50° C.
Relative Humidity: Start: 45.03%, 15 minutes: 62.93%, 3 hours: 66.65%

One carrier for each exposure time was each placed at the low height, middle height and high height in the testing room. The distance from the device were as follows: Low: ˜5 ft. Middle: ˜6.6 ft. High: ˜12.4 ft. The device was operated with INTERVAL set to 30 minutes and DURATION set to 5 seconds. No adjustment was required during testing. The haze level in testing room was maintained at moderate haze level per (from a distance of ˜10 feet) for the specified exposure times.

### Recovery of Virus Following Exposure

Following exposure, a 2.0 mL aliquot of test medium was added to each carrier and scraped with a cell scraper to resuspend the contents (10-1 dilution). The test medium was collected, passed through individually prepared Sephadex columns and then serial 10-fold dilutions were performed. Each dilution was then assayed for infectivity and/or cytotoxicity.

### Dried Virus Control

The appropriate number of virus films (zero time and both exposure times, in triplicate) were prepared as described previously and were run in parallel to the test virus. Each virus control film was held covered for the same exposure time and at the same exposure temperature as the test films. Immediately following the exposure, a 2.0 mL aliquot of test medium was added to the carrier and scraped with a cell scraper to resuspend the contents (10-1 dilution). The test medium was collected, passed through individually prepared Sephadex columns, and then serial 10-fold dilutions were performed. Each dilution was assayed for infectivity.

### Cytotoxicity Control

A carrier was dried as above, using an aliquot of test medium containing the requested organic soil load in lieu of virus. Following drying, the carrier was exposed to the test device in parallel with the test carriers (for the longest exposure time). Following exposure, the recovery was the same as indicated above in testing. Serial 10-fold dilutions were then performed and each dilution was assayed for cytotoxicity. The placement location of the carrier in the testing room was at the middle height.

### Assay of Non-Virucidal Level of Test Substance (Neutralization Control)

Each dilution of the neutralized test substance (cytotoxicity control dilutions) was challenged with an aliquot of low titer stock virus to determine the dilution(s) of test substance at which virucidal activity, if any, is retained. Dilutions that show virucidal activity were not be considered in determining reduction of the virus by the test disinfecting composition.

Using the cytotoxicity control dilutions prepared above, an additional set of indicator cell cultures was inoculated with a 100 μL aliquot of each dilution in quadruplicate. A 100 μL aliquot of low titer stock virus was inoculated into each cell culture well and the indicator cell cultures were incubated along with the test and virus control plates.

### Test Results

Under the conditions of this study and in the presence of a 1% fetal bovine serum organic soil load, the test disinfection composition (which is ready to use) demonstrated reductions in viral titer of Human Coronavirus following 15 minute and 3 hour exposure times. The average titer of the dried virus controls was 4.50 Log₁₀/100 μL at time zero, 15 minutes, and 3 hours as the virus remained relatively stable on the surface and was not affected by the environment during this period of time. These results are summarized in Table 1.

TABLE 1

| Exposure | | Carrier | | Log | Overall Log |
|---|---|---|---|---|---|
| Time | | Height | Test Results¹ | Reduction² | Reduction³ |
| 15 | minutes | Low | 3.50 Log₁₀/100 μL | 1.00 Log₁₀ | 0.49 Log₁₀ |
| | | Middle | 4.00 Log₁₀/100 μL | 0.50 Log₁₀ | |
| | | High | 4.25 Log₁₀/100 μL | 0.25 Log₁₀ | |
| 3 | hours | Low | 2.25 Log₁₀/100 μL | 2.25 Log₁₀ | 2.15 Log₁₀ |
| | | Middle | 2.50 Log₁₀/100 μL | 2.00 Log₁₀ | |
| | | High | 2.25 Log₁₀/100 μL | 2.25 Log₁₀ | |

¹The average titer of virus (i.e., the amount of virus as determined by titration) at the exposure time in test chamber.
²Log reduction was calculated by deducting the average titer of virus obtained from each type of carrier at the specified exposure time from the average titer of the dried virus controls at the same exposure time.
³Overall log reduction was calculated by deducting the average titer of virus obtained from all of the carriers (i.e., all of the low, middle, and high carriers) at the specified exposure time from the average titer of the dried virus controls at the same exposure time.

As shown in Table 1, the test disinfecting composition unexpectedly exhibited average Log₁₀ viral reduction of at least 0.49 and 2.15 after an exposure time of 15 minutes and 3 hours against human coronavirus, respectively. In other words, the composition was able to kill more than 99% of the human coronavirus on a hard surface in 3 hours.

### Example 2: Evaluation of a Disinfecting Composition for its Efficacy Against a Non-Enveloped Virus on a Hard Non-Porous Surface

The disinfecting composition described in Example 1 was also tested for its efficacy against a non-enveloped virus using the same procedures described in Example 1. The test parameters used in this example are listed below.

### Test Parameters

Dilution of Disinfecting Composition: No dilution.

### Virus: Human Adenovirus Type 5, ATCC VR-5, Strain Adenoid 75

Exposure Times: 15 minutes and 3 hours
Exposure Temperature: 25-29° C. (prior to use in testing, the room was brought to temp before turning off the air handling system, Start: 24.73° C., 15 minutes: 24.63° C., 3 hours: 23.75° C.)
Exposure Humidity: Start: 54.38%, 15 minutes: 43.29%, 3 hours: 48.10%
Organic Soil Load: 1% fetal bovine serum
Test Medium: Minimum Essential Medium (MEM) supplemented with 5% (v/v) heat-inactivated fetal bovine serum, 100 units/ml penicillin, 10 μg/ml gentamicin, and 2.5 μg/ml amphotericin B.

Indicator Cell Cultures: A-549 (human lung carcinoma) cells.

In addition, the amounts of the test disinfecting composition at various stages are listed below.

### Pre Test: 4092.17 g

### Post Prime: 4086.59 g

### Post Test: 3994.61 g

### Test Results

Under the conditions of this study and in the presence of a 1% fetal bovine serum organic soil load, the test disinfection composition (which is ready to use) demonstrated reductions in viral titer of Human Adenovirus type 5 following 15 minute and 3 hour exposure times. Due to natural dying off, the average titer of the dried virus controls were 7.33 Log₁₀/100 μL, 5.64 Log₁₀/100 μL, and 5.85 Log₁₀/100 μL at time zero, 15 minutes, and 3 hours, respectively. These results are summarized in Table 2.

TABLE 2

| Exposure | | Carrier | | Log | | Overall Log |
|---|---|---|---|---|---|---|
| Time | | Height | Test Results¹ | Reduction² | | Reduction³ |
| 15 | minutes | Low | 5.25 Log₁₀/100 μL | 0.39 | Log₁₀ | 0.04 Log₁₀ |
| | | Middle | 4.50 Log₁₀/100 μL | 1.14 | Log₁₀ | |
| | | High | 6.00 Log₁₀/100 μL | No reduction | | |
| 3 | hours | Low | 4.25 Log₁₀/100 μL | 0.6 | Log₁₀ | 1.84 Log₁₀ |
| | | Middle | 3.50 Log₁₀/100 μL | 2.35 | Log₁₀ | |
| | | High | 4.00 Log₁₀/100 μL | 1.85 | Log₁₀ | |

¹The average titer of virus (i.e., the amount of virus as determined by titration) at the exposure time in test chamber.
²Log reduction was calculated by deducting the average titer of virus obtained from each type of carrier at the specified exposure time from the average titer of the dried virus controls at the same exposure time.
³Overall log reduction was calculated by deducting the average titer of virus obtained from all of the carriers (i.e., all of the low, middle, and high carriers) at the specified exposure time from the average titer of the dried virus controls at the same exposure time.

As shown in Table 2, the test disinfecting composition unexpectedly exhibited a Log₁₀ viral reduction of at least 1.84 after an exposure time of 3 hours against human adenovirus.

### Example 3: Evaluation of a Disinfecting Composition for its Efficacy Against Non-Enveloped Virus MS2 Bacteriophage (MS2) in an Aerosol

MS2 Bacteriophage (MS2), ATCC 15597-B1 was selected for this test. This virus is a non-enveloped positive-stranded RNA virus of the bacteriophage family Leviviridae. Bacterial cells are the hosts for bacteriophages, and E. coli 15597 served this purpose for MS2 bacteriophage. Its small size, icosohedral structure, and environmental resistance has made MS2 ideal for use as a surrogate virus (particularly in place of picornaviruses such as poliovirus and human norovirus) in water quality and disinfectant studies.

MS2 was grown on appropriate media. The culture used for test inoculum are evaluated for sterility, washed and concentrated in sterile phosphate buffered saline upon harvesting. Virus concentrations were determined after incubation at 36±1° C. for 18-24 hours. MS2 samples were enumerated in 50% tryptic soy agar using standard dilution and plating techniques. The test inoculum was split into two equal parts and added to the appropriate number of nebulizers. Liquid culture should not exceed 20 ml per nebulizer. The testing parameters are as follows:

Volume of inoculum added to nebulizer: 20 ml
Sampler Media (Volume): Phosphate buffered saline with 0.1% Tween 80 (20 ml)
Sampling Time: 10 minutes
Sampling Type: Impingers, SKC biosamplers

### Incubation Temperature: 36±1° C.

Nebulization Time: 60 minutes
Neck Rinse Media (Volume): Phosphate buffered saline (5 ml)
Disinfecting Composition Contact Times: 0, 10 minutes, 1.5 hours, and 3 hours

### Enumeration Media: 50% Tryptic Soy Agar

Incubation Time: 18-24 hours

Hurricane 1800 Flex (Chauvet DJ, Sunrise, Fla.) was used as a fog generator/nebulizer and was setup as described in Example 1 above. The test disinfecting composition contained triethylene glycol (52.25 wt %), propylene glycol (1 wt %), and DI water (46.75%). 511 g of the test composition was weighed out into the fog fluid container of Hurricane 1800 Flex and the device was primed as described in Example 1. The test composition still in the Hurricane 1800 Flex was weighed after the priming and there was 492 g remaining. The test composition was weighed again after the study was completed and there was 464 g remaining.

The testing chamber was setup and the safety checklist was completed prior to test initiation. Tests were initiated by aerosolizing the MS2 using the nebulizers for 60 minutes and allowing the concentration to reach the required PFU/m³. In a baseline run, no test composition was added to the testing chamber and samples were taken at 1 hour, 2 hours, and 3 hours to determine virus die off and settling at these times. In a test run, once the required viral concentration was reached in the testing chamber, a time zero sample was taken. The Hurricane 1800 Flex was then run for the specified contact time (i.e., 10 minutes, 1.5 hours, and 3 hours) and an additional sample was taken at each contact time. All samples were taken in phosphate buffered saline with 0.1% Tween 80 (20 ml) for 10 minutes by using an SKC Biosampler®.

Once the test was completed, the decontamination process was performed by using 4 hours of UV exposure prior to any scientist entering the testing chamber. Reductions of MS2 were calculated relative to its concentration at the time zero or corresponding control run sample as applicable. The test result at 10 minute contact time was compared with the result at time zero of the baseline run as the test microorganism's natural die off and settling at 10 minute is negligible.

The viral concentration in the testing chamber was calculated using the following equations:

PFU/ml=(Average plate count)×1:10 serial dilution factor

PFU/m³=[(PFU/ml×Vₛ)÷(Tₛ×12.5 L/min)]×(1000 L/m³)

in which Vₛ=Bio-sampler volume (ml) and Tₛ=Time sampled (min). In addition, the reduction in the viral concentration is calculated by the following equation:

Log₁₀ Reduction=Log(B/A)

in which B=Number of viable test microorganisms on the control carriers immediately after inoculation; and A=Number of viable test microorganisms on the test carriers after the contact time.

The test results are summarized in Table 3 below.

TABLE 3¹

| | | | | | | Log₁₀ | Log₁₀ |
|---|---|---|---|---|---|---|---|
| | | | | | | Reduction | Reduction²⁻⁴ |
| Test | Run | Treatment | | | Average | Compared | Compared |
| Microorganism | Type | Time Point | Replicate | PFU/m³ | PFU/m³ | to Time Zero | to Baseline |

| MS2 | Baseline | Time Zero | Replicate 1 | 1.58E+04 | 1.47E+04 | N/A |
|---|---|---|---|---|---|---|
| Bacteriophage | | | Replicate 2 | 1.32E+04 | | |
| ATCC 15597-B1 | | | Replicate 3 | 1.51E+04 | | |

| | | | | | | |
|---|---|---|---|---|---|---|
| 1 | hour | Replicate 1 | 1.02E+03 | 1.92E+03 | 0.88 | N/A |
| | | Replicate 2 | 2.62E+03 | | | |
| | | Replicate 3 | 2.13E+03 | | | |
| 2 | hours | Replicate 1 | 1.28E+03 | 1.18E+03 | 1.10 | N/A |
| | | Replicate 2 | 9.12E+02 | | | |
| | | Replicate 3 | 1.34E+03 | | | |
| 3 | hours | Replicate 1 | 8.57E+02 | 7.18E+02 | 1.31 | N/A |
| | | Replicate 2 | 5.81E+02 | | | |
| | | Replicate 3 | 7.17E+02 | | | |

| | | | | | |
|---|---|---|---|---|---|
| Test | Time Zero | Replicate 1 | 1.12E+05 | 1.18E+05 | N/A |

| | | Replicate 2 | 1.23E+05 | | | |
|---|---|---|---|---|---|---|
| | | Replicate 3 | 2.16E+05 | | | |
| 10 | minutes³ | Replicate 1 | <8.80E+01 | <8.96E+01 | >3.12 | >3.12 |
| | | Replicate 2 | <9.12E+01 | | | |
| | | Replicate 3 | <8.96E+01 | | | |
| 1.5 | hours | Replicate 1 | <9.12E+01 | <8.92E+01 | >3.12 | >2.02 |
| | | Replicate 2 | <8.72E+01 | | | |
| | | Replicate 3 | <9.04E+01 | | | |
| 3 | hours | Replicate 1 | <8.80E+01 | <8.92E+01 | >3.13 | >1.82 |
| | | Replicate 2 | <8.72E+01 | | | |
| | | Replicate 3 | <9.12E+01 | | | |

¹The limit of detection for this assay is 8.72E+01 PFU/m³ and values below the limit of detection are noted as “<8.72E+01” in the data table.
²The Log reductions for the Test Runs were adjusted to account for natural die-off and gravitational settling observed in the Baseline Runs.
³The result at 10-minute contact time was compared with the results at the time zero of the baseline run as the test microorganism's natural die off and settling at 10 minute is negligible.
⁴The results at 1.5-hour and 3-hour contact times were compared with the results at 2-hour and 3-hour of the baseline runs, respectively.

As shown in Table 3, the test disinfecting composition exhibited a log₁₀ Reduction as high as at least 3.12 in 10 minutes compared to the baseline results. In other words, the test disinfecting composition was able to kill at least 99.9% of MS2 in 10 minutes after accounting for the natural die off of the virus. Note that the log₁₀ Reduction values compared to baseline at 1.5 hours and 3 hours became lower than the log₁₀ Reduction value at 10 minutes because the viral concentrations at 1.5 hours and 3 hours were below detection limit in the test runs, while the viral concentrations at corresponding time in baseline runs were falling as time increased due to natural dying off and settling.

### Example 4: Evaluation of a Disinfecting Composition for its Efficacy Against Non-Enveloped Virus MS2 Bacteriophage (MS2) in an Aerosol

The disinfecting composition used in Example 3 was tested against MS2 Bacteriophage following a procedure similar to that described in Example 3 where the main differences include: (1) a smaller amount of the disinfecting composition was used (which resulted in a lower concentration of the composition in the air of the testing chamber), (2) the viral concentration in the testing chamber was measured at time zero, 3 minutes, 15 minutes, and 27 minutes (instead of time zero, 10 minutes, 1.5 hours, and 3 hours). The testing parameters are as follows:

Volume of inoculum added to nebulizer: 20 ml
Sampler Media (Volume): Phosphate buffered saline with 0.1% Tween 80 (20 ml)
Sampling Time: 10 minutes
Sampling Type: Impingers, SKC biosamplers

### Incubation Temperature: 36±1° C.

Nebulization Time: 60 minutes
Neck Rinse Media (Volume): Phosphate buffered saline (5 ml)
Disinfecting Composition Contact Times: 0, 3 minutes, 15 minutes, and 27 minutes

### Enumeration Media: 50% Tryptic Soy Agar

Incubation Time: 12-18 hours

The test results are summarized in Table 4 below.

TABLE 4

| | | | | | | Percentage | Log₁₀ | Log₁₀ |
|---|---|---|---|---|---|---|---|---|
| | | | | | | Reduction | Reduction | Reduction |
| Test | Run | Treatment | | | Average | Compared | Compared | Compared |
| Microorganism | Type | Time Point | Replicate | PFU/m³ | PFU/m³ | to Time Zero | to Time Zero | to Baseline |

| | | | | | | | |
|---|---|---|---|---|---|---|---|
| MS2 | Baseline | Time Zero | Replicate 1 | 3.30E+05 | 4.06E+05 | N/A | N/A |
| Bacteriophage | | | Replicate 2 | 4.83E+05 | | | |
| ATCC 15597-B1 | | | Replicate 3 | 4.26E+05 | | | |

| | | | | | | | |
|---|---|---|---|---|---|---|---|
| 3 | minutes | Replicate 1 | 4.14E+05 | 3.42E+05 | 15.81% | 0.07 | N/A |
| | | Replicate 2 | 2.70E+05 | | | | |
| | | Replicate 3 | 2.83E+05 | | | | |
| 15 | minutes | Replicate 1 | 1.53E+05 | 1.66E+05 | 59.22% | 0.39 | N/A |
| | | Replicate 2 | 1.78E+05 | | | | |
| | | Replicate 3 | 1.07E+05 | | | | |
| 27 | minutes | Replicate 1 | 7.84E+04 | 8.16E+04 | 79.90% | 0.7 | N/A |
| | | Replicate 2 | 8.48E+04 | | | | |
| | | Replicate 3 | 5.06E+04 | | | | |

| | | | | | | |
|---|---|---|---|---|---|---|
| Test | Time Zero | Replicate 1 | 3.05E+05 | 3.39E+05 | N/A | N/A |
| | | Replicate 2 | 3.73E+05 | | | |
| | | Replicate 3 | 7.39E+05 | | | |

| | | | | | | | |
|---|---|---|---|---|---|---|---|
| 3 | minutes | Replicate 1 | 8.14E+02 | 8.10E+02 | 99.76% | 2.62 | 2.55 |
| | | Replicate 2 | 8.06E+02 | | | | |
| | | Replicate 3 | 1.78E+02 | | | | |
| 15 | minutes | Replicate 1 | 9.28E+01 | <1.35E+02 | >99.96% | >3.4 | >3.01 |
| | | Replicate 2 | 1.78E+02 | | | | |
| | | Replicate 3 | <8.72E+01 | | | | |
| 27 | minutes | Replicate 1 | <9.12E+01 | <9.08E+01 | >99.97% | >3.57 | >2.88 |
| | | Replicate 2 | <9.04E+01 | | | | |
| | | Replicate 3 | <9.12E+01 | | | | |

As shown in Table 4, the test disinfecting composition exhibited a log₁₀ Reduction as high as 2.55 in 3 minutes compared to the baseline results. In other words, the test disinfecting composition was able to kill at least 99% of MS2 in 3 minutes after accounting for the natural die off the virus.

### Example 5: Evaluation of a Disinfecting Composition for its Efficacy Against Non-Enveloped Virus MS2 Bacteriophage (MS2) in an Aerosol

The disinfecting composition used in Example 3 was tested against MS2 Bacteriophage following a procedure to that described in Example 4 where the main differences include that (1) smaller amounts (i.e., 1 g or 3 g) of the disinfecting composition was used (which resulted in a lower concentration of the composition in the air of the testing chamber), (2) the initial viral concentration was increased (i.e., to 8.3×10⁷-9.75×10⁸ PFU/m³), and (3) the viral concentration in the testing chamber was measured at time zero, 30 seconds, 15 minutes, and 60 minutes (instead of time zero, 10 minutes, 1.5 hours, and 3 hours).

In particular, the disinfecting composition was tested at two different levels: (a) non-visual haze level (i.e., using a total of 1 g during the 60-minute test), which is referred to as Test 1 and (b) very light haze level (i.e., using a total of 3 g during the entire 60-minute test), which is referred to as Test 2. Test 1 was performed by dosing the test chamber with the disinfecting composition every 10 minutes for 10 seconds to achieve a non-visual haze level in the chamber. Test 2 was performed by dosing the test chamber with the disinfecting composition every 30 minutes for 30 seconds to achieve a very light haze level in the chamber.

To measure the concentrations of the disinfecting composition and TEG in the test chamber at different time points, the disinfecting composition alone (without MS2 Bacteriophage) was dosed into the test chamber in a separate test. This test was performed by dosing the test chamber with the disinfecting composition immediately after time zero for 10 seconds and then in every 20 minutes for 10 seconds at 30% RH. The concentrations of the disinfecting composition at different time points were measured by using TSI DUSTTRAK™ DRX 8534 Aerosol Monitor (“TSI 8534 Monitor”), which was calibrated to Standard 12103-1 A1 utilizing “Ultrafine Arizona Road Dust”. The results are summarized in Tables 5 and 6 below.

In Tables 5 and 6, “measured conc. of disinfecting composition” refers to the concentrations measured directly from the sensor on the TSI 8534 Monitor. However, when measuring any material other than Arizona Road Dust, a calibration factor must be applied and is calculated as follows. Arizona Road dust has a density of 2.7 g/cm³, while the tested disinfecting composition has a density of 1.08 g/cm³. This density difference needs to be corrected for and results in a correction factor of 0.4 (i.e., 1.08/2.7=0.4). As an example, if the sensor on the TSI 8534 Monitor has a reading of 2 mg/m³ as the measured concentration of the disinfecting composition, the actual concentration of the tested disinfecting composition in the air would be calculated as 0.8 mg/m³ (i.e., 0.4×2 mg/m³=0.8 mg/m³). Based on this correction factor, the actual concentrations of the tested disinfecting composition at different time points were calculated and summarized in Tables 5 and 6 under “actual conc. of disinfecting composition”.

In addition, the actual concentration of total triethylene glycol (TEG) in the air were calculated as follows based on NIOSH 5523 test. Specifically, the average recorded concentrations of the tested disinfecting composition from the sensor on the TSI 8534 Monitor at different levels were plotted against laboratory measured concentrations of total TEG, and a linear regression model was used to show the relationship between the two concentration values. The slope of the curve was calculated to determine the correction factor, which was 0.25. As an example, if the sensor on the TSI 8534 Monitor has a reading of 2 mg/m³ as the measured concentration of the disinfecting composition, the actual concentration of the TEG in the air would be calculated as 0.5 mg/m³ (i.e., 0.25×2 mg/m³=0.5 mg/m³). Based on this correction factor, the actual concentrations of the TEG at different time points were calculated and summarized in Table 7 under “actual conc. of TEG”.

TABLE 5
Concentration of disinfecting composition at non-visual haze level

| | Measured conc. | Actual conc. | |
|---|---|---|---|
| | of disinfecting | of disinfecting | Actual conc., |
| | composition | composition | of TEG |
| Time Point | (mg/m³) | (mg/m³) | (mg/m³) |
| Time zero* | 0.406 | 0.162 | 0.102 |
| 30 seconds | 4.11 | 1.64 | 1.03 |
| 15 minutes | 6.74 | 2.70 | 1.69 |
| 60 minutes | 1.72 | 0.69 | 0.43 |

*Time zero data were obtained by measuring any background particles in the chamber before disinfecting composition was introduces.

TABLE 6
Concentration of disinfecting composition at very light haze level

| | Measured conc. | Actual conc. | |
|---|---|---|---|
| | of disinfecting | of disinfecting | Actual conc. |
| | composition | composition | of TEG |
| Time Point | (mg/m³) | (mg/m³) | (mg/m³) |
| Time zero | 0.004 | 0.0016 | 0.001 |
| 30 seconds | 10.7 | 4.28 | 2.68 |
| 15 minutes | 14.2 | 5.68 | 3.55 |
| 60 minutes | 6.89 | 2.76 | 1.72 |

To measure the efficacy of the disinfecting composition in reducing the viral concentration in the test chamber at different time points, MS2 Bacteriophage was first introduced into the test chamber and initial concentration was measured at time zero. The disinfecting composition was dosed into the test chamber immediately after time zero for 10 seconds and then every 10 minutes for 10 seconds at 30% RH for Test 1 (non-visual haze level) and for 20 seconds and then every 30 minutes for 20 seconds at 30% RH for Test 2 (very light haze level). The efficacy results of the tested disinfection composition are summarized in Table 7 below.

TABLE 7

| | | | | | | | | |
|---|---|---|---|---|---|---|---|---|
| | | | | | | Percentage | Log₁₀ | Log₁₀ |
| | | | | | | Reduction | Reduction | Reduction |
| Test | Run | Treatment | | | Average | Compared | Compared | Compared |
| Microorganism | Type | Time Point | Replicate | PFU/m³ | PFU/m³ | to Time Zero | to Time Zero | to Baseline |

| | | | | | | | |
|---|---|---|---|---|---|---|---|
| MS2 | Baseline | Time Zero | Replicate 1 | 6.39E+08 | 7.79E+08 | N/A | N/A |
| Bacteriophage | | | Replicate 2 | 7.24E+08 | | | |
| ATCC 15597-B1 | | | Replicate 3 | 9.75E+08 | | | |

| | | | | | | |
|---|---|---|---|---|---|---|
| 30 seconds | Replicate 1 | 7.02E+08 | 7.26E+08 | 6.890% | 0.03 | N/A |
| | Replicate 2 | 9.00E+08 | | | | |
| | Replicate 3 | 5.76E+08 | | | | |
| 15 minutes | Replicate 1 | 3.65E+08 | 3.52E+08 | 54.857% | 0.35 | N/A |
| | Replicate 2 | 3.78E+08 | | | | |
| | Replicate 3 | 3.12E+08 | | | | |
| 60 minutes | Replicate 1 | 5.24E+07 | 4.64E+07 | 94.044% | 1.23 | N/A |
| | Replicate 2 | 3.61E+07 | | | | |
| | Replicate 3 | 5.08E+07 | | | | |

| | | | | | | |
|---|---|---|---|---|---|---|
| Test 1 | Time Zero | Replicate 1 | 1.21E+08 | 1.45E+08 | N/A | N/A |
| Non-visual | | Replicate 2 | 1.28E+08 | | | |
| Haze Level | | Replicate 3 | 1.86E+08 | | | |

| | | | | | | |
|---|---|---|---|---|---|---|
| 30 seconds | Replicate 1 | 1.66E+06 | 2.59E+06 | 98.21% | 1.75 | 1.72 |
| | Replicate 2 | 3.59E+06 | | | | |
| | Replicate 3 | 2.53E+06 | | | | |
| 15 minutes | Replicate 1 | 6.86E+04 | 5.51E+04 | 99.96% | 3.42 | 3.07 |
| | Replicate 2 | 4.75E+04 | | | | |
| | Replicate 3 | 4.93E+04 | | | | |
| 60 minutes | Replicate 1 | 1.50E+04 | 1.41E+04 | 99.9903% | 4.01 | 2.79 |
| | Replicate 2 | 1.57E+04 | | | | |
| | Replicate 3 | 1.16E+04 | | | | |

| | | | | | | |
|---|---|---|---|---|---|---|
| Test 2 | Time Zero | Replicate 1 | 8.30E+07 | 9.71E+07 | N/A | N/A |
| Very Light | | Replicate 2 | 8.81E+07 | | | |
| Haze Level | | Replicate 3 | 1.20E+08 | | | |

| | | | | | | |
|---|---|---|---|---|---|---|
| 30 seconds | Replicate 1 | 1.48E+05 | 1.45E+05 | 99.85% | 2.83 | 2.80 |
| | Replicate 2 | 1.97E+05 | | | | |
| | Replicate 3 | 8.80E+04 | | | | |
| 15 minutes | Replicate 1 | 5.10E+04 | 4.84E+04 | 99.95% | 3.30 | 2.96 |
| | Replicate 2 | 5.46E+04 | | | | |
| | Replicate 3 | 3.96E+04 | | | | |
| 60 minutes | Replicate 1 | 1.28E+04 | 1.15E+04 | 99.988% | 3.93 | 2.70 |
| | Replicate 2 | 1.07E+04 | | | | |
| | Replicate 3 | 1.12E+04 | | | | |

As shown in Table 7, the test disinfecting composition exhibited a log₁₀ Reduction as high as 1.72 in 30 seconds at the non-visual haze level and 2.80 in 30 seconds at the very light haze level compared to the baseline results. In other words, the test disinfecting composition was able to kill at least 98% of MS2 in 30 seconds at the non-visual haze level and at least 99.8% of MS2 in 30 seconds at the very light haze level after accounting for the natural die off the virus.

### Example 6: Evaluation of a Disinfecting Composition for its Efficacy at a Low Concentration Against Non-Enveloped Virus MS2 Bacteriophage (MS2) in an Aerosol

The disinfecting composition used in Example 3 was tested against MS2 Bacteriophage following a procedure to that described in Example 5 where the main differences include that (1) smaller amounts (i.e., less than 1 g) of the disinfecting composition were used (which resulted in a lower concentration of the composition in the air of the testing chamber), and (2) the initial viral concentration was increased (i.e., to 1.11-1.94×10⁹ PFU/m³). The viral concentration in the testing chamber was still measured at time zero, 30 seconds, 15 minutes, and 60 minutes. The fog generator used in this example was an Amhaze Stadium haze machine (instead of Hurricane 1800 FLEX).

In particular, the disinfecting composition was tested at a low concentration level, i.e., using a total of less than 1 g during the 60-minute test. To measure the concentrations of the disinfecting composition and TEG in the test chamber at different time points, the disinfecting composition alone (without MS2 Bacteriophage) was dosed into the test chamber in a separate test. This test was performed by dosing the test chamber with the disinfecting composition immediately after time zero for 10 seconds and then every 20 minutes for 10 seconds at 30% RH. The concentrations of the disinfecting composition at different time points were measured by using TSI DUSTTRAK™ DRX 8534 Aerosol Monitor (“TSI 8534 Monitor”) using the same procedure described in Example 5 and are summarized in Table 8 below. The actual concentration of disinfecting composition and actual concentration TEG were calculated using the same methods described in Example 5 and are summarized in Table 8 below.

TABLE 8

| | Measured Conc. | Actual Conc. | |
|---|---|---|---|
| | of disinfecting | of disinfecting | Actual Conc. |
| | composition | composition | of TEG |
| Time Point | (mg/m³) | (mg/m³) | (mg/m³) |
| Time zero | 0.012 | 0.0048 | 0.003 |
| 30 seconds | 2.33 | 0.932 | 0.582 |
| 15 minutes | 0.019 | 0.0076 | 0.004 |
| 60 minutes | 6.09 | 2.436 | 1.522 |

To measure the efficacy of the disinfecting composition in reducing the viral concentration in the test chamber at different time points, MS2 Bacteriophage was first introduced into the test chamber and initial concentration was measured at time zero. The disinfecting composition was dosed into the test chamber immediately after time zero for 10 seconds and then every 20 minutes for 10 seconds at 30% RH. The efficacy results of the tested disinfection composition are summarized in Table 9 below.

TABLE 9

| | | | | | | | | |
|---|---|---|---|---|---|---|---|---|
| | | | | | | Percentage | Log₁₀ | Log₁₀ |
| | | | | | | Reduction | Reduction | Reduction |
| Test | Run | Treatment | | | Average | Compared | Compared | Compared |
| Microorganism | Type | Time Point | Replicate | PFU/m³ | PFU/m³ | to Time Zero | to Time Zero | to Baseline |

| | | | | | | | |
|---|---|---|---|---|---|---|---|
| MS2 | Baseline | Time Zero | Replicate 1 | 1.21E+09 | 1.25E+09 | N/A | N/A |
| Bacteriophage | | | Replicate 2 | 1.43E+09 | | | |
| ATCC 15597-B1 | | | Replicate 3 | 1.11E+09 | | | |

| | | | | | | |
|---|---|---|---|---|---|---|
| 30 seconds | Replicate 1 | 1.10E+09 | 1.20E+09 | 4.33% | 0.02 | N/A |
| | Replicate 2 | 1.18E+09 | | | | |
| | Replicate 3 | 1.31E+09 | | | | |
| 15 minutes | Replicate 1 | 8.95E+08 | 9.55E+08 | 23.57% | 0.12 | N/A |
| | Replicate 2 | 9.50E+08 | | | | |
| | Replicate 3 | 1.02E+08 | | | | |
| 60 minutes | Replicate 1 | 5.90E+08 | 5.42E+08 | 56.62% | 0.36 | N/A |
| | Replicate 2 | 5.80E+08 | | | | |
| | Replicate 3 | 4.58E+08 | | | | |

| | | | | | | |
|---|---|---|---|---|---|---|
| Test | Time Zero | Replicate 1 | 1.83E+09 | 1.77E+09 | N/A | N/A |
| 30% RH | | Replicate 2 | 1.94E+09 | | | |
| | | Replicate 3 | 1.53E+09 | | | |

| | | | | | | |
|---|---|---|---|---|---|---|
| 30 seconds | Replicate 1 | 2.74E+07 | 1.86E+07 | 98.95% | 1.98 | 1.96 |
| | Replicate 2 | 1.54E+07 | | | | |
| | Replicate 3 | 1.29E+07 | | | | |
| 15 minutes | Replicate 1 | 9.03E+06 | 8.44E+06 | 99.52% | 2.32 | 2.20 |
| | Replicate 2 | 7.86E+06 | | | | |
| | Replicate 3 | 8.44E+06 | | | | |
| 60 minutes | Replicate 1 | 1.58E+06 | 1.37E+06 | 99.92% | 3.11 | 2.75 |
| | Replicate 2 | 1.04E+06 | | | | |
| | Replicate 3 | 1.49E+06 | | | | |

As shown in Table 9, the test disinfecting composition exhibited a log₁₀ Reduction as high as 1.96 in 30 seconds compared to the baseline results. In other words, the test disinfecting composition was able to kill about 99% of MS2 in 30 seconds after accounting for the natural die off the virus.

Other embodiments are in the following claims.

1. A method for disinfecting a space, comprising:
spraying a composition comprising triethylene glycol into a space containing severe acute respiratory syndrome coronavirus 2 (SARS-CoV-2) in an amount effective to inactivate SARS-CoV-2.

2. The method of claim 1, wherein the triethylene glycol is in an amount of from about 10% to about 90% by weight of the composition.

3. The method of claim 1, wherein the composition further comprises deionized water.

4. The method of claim 3, wherein the deionized water is in an amount of from about 5% to about 90% by weight of the composition.

5. The method of claim 1, wherein the composition further comprises propylene glycol or polyethylene glycol.

6. The method of claim 5, wherein the propylene glycol is in an amount of from about 0.5% to about 5% by weight of the composition.

7. The method of claim 1, wherein the composition comprises from about 50% to about 90% by weight triethylene glycol, and from about 10% to about 50% by weight deionized water.

8. The method of claim 1, wherein the composition comprises about 52.25% by weight triethylene glycol, about 1% by weight propylene glycol, and about 46.75% by weight deionized water.

9. The method of claim 1, wherein spraying the composition forms an aerosol.

10. The method of claim 9, wherein the aerosol comprises liquid droplets having an average diameter of from about 10 nm to about 10 μm.

11. The method of claim 1, further comprising vaporizing the composition before spraying the composition.

12. The method of claim 11, wherein vaporizing the composition is performed by treating the composition with ultrasonication, atomization, steam, or heating.

13. The method of claim 1, wherein the space comprises SARS-CoV-2 suspending in the air and the method is capable of inactivating the SARS-CoV-2 in the air.

14. The method of claim 1, wherein the space comprises SARS-CoV-2 on a surface and the method is capable of inactivating the SARS-CoV-2 on the surface.

15. The method of claim 1, wherein the space is an indoor space.

16. The method of claim 1, wherein the method is performed by a humidifier or a smoke generator.

17. A composition, comprising:
triethylene glycol in an amount of from about 52% to about 90% by weight of the composition;
deionized water in an amount of from about 5% to about 48% by weight of the composition; and
propylene glycol in an amount of from about 0% to about 5% by weight of the composition.

18. The composition of claim 17, wherein the composition comprises:
triethylene glycol in an amount of from about 52% to about 90% by weight of the composition;
deionized water in an amount of from about 5% to about 47.5% by weight of the composition; and
propylene glycol in an amount of from about 0.5% to about 5% by weight of the composition.

19. The composition of claim 18, wherein the composition consists of:
triethylene glycol in an amount of from about 52% to about 90% by weight of the composition;
deionized water in an amount of from about 5% to about 47.5% by weight of the composition; and
propylene glycol in an amount of from about 0.5% to about 5% by weight of the composition.

20. The composition of claim 19, wherein the composition consists of:
about 52.25% by weight triethylene glycol;
about 46.75% by weight deionized water; and
about 1% by weight propylene glycol.

21. A packaged product, comprising:
a container; and
the composition of claim 17 in the container.
